(19) United States
(12) Patent Application Publication
(10) Pub. No.: US 2020/0205492 A1
(43) Pub. Date: Jul. 2, 2020

(54) Surgical Gown, Gloves, and Cap With Cooling Properties

(71) Applicant: Medline Industries, Inc., Northfield, IL (US)

(72) Inventors: Mark Spencer Chua, Northbrook, IL (US); Michael Turturro, Arlington Heights, IL (US); Lynn Gao, Shanghai (CN); Min Yao, Libertyville, IL (US); Komal Pokharel-adhikari, Mundelein, IL (US)

(21) Appl. No.: 16/232,531

(22) Filed: Dec. 26, 2018

(51) Int. Cl. A41D 13/12; A41D 31/02; A41D 19/00; A42B 1/00; A42B 1/04; A61B 42/10

(52) U.S. Cl. CPC A41D 13/1209; A41D 13/129; A41D 31/02; A41D 19/0006; A42B 1/008; A41D 2600/20; A61B 42/10; A41D 2300/33; A41D 2500/30; A41D 2500/54; A42B 1/043

(57) ABSTRACT

In some embodiments, apparatuses and methods are presented herein useful to provide a protective medical garment, such as a gown, head covering, or gloves with a cooling property or agent. In one approach, the protective medical garment comprises a non-woven fabric that is at least partially-treated on an interior surface thereof with a phase change cooling agent. For example, a surgical gown may have a torso section and sleeves, with the phase change cooling agent deposited in areas adjacent the wearer's trunk, neck, underarms, or wrists.

### TECHNICAL FIELD

The present disclosure relates generally to the field of hospital protective garments, and more particularly to surgical gowns.

### BACKGROUND

Protective medical clothing, such as gowns, head coverings or caps, and gloves, are used for a variety of medical activities. Surgical gowns and caps are generally employed with water-repellent and water-impermeable barrier layers, even though these layers, in some configurations, make the garment less breathable.

Further, such clothing items may be worn for a significant amount of time, especially during particularly complicated or lengthy surgical procedures. This can lead to heat discomfort for the surgical team, particularly at the typical operating room temperatures of 19° C. to 20° C. and typical relative humidity of 70%. During periods of high stress or high activity level these concerns can be exacerbated. Thus, improvements to the comfort of the medical garments worn in the operating theater are of interest to surgeons.

It has now been found that phase change materials may be incorporated into surgical clothing, including for example non-woven surgical clothing such as spunbond-meltblown-spunbond fabrics. Such phase change materials can assist in cooling the body temperature of surgical staff via the mechanisms described herein.

### BRIEF DESCRIPTION OF THE DRAWINGS

FIG. 1 is a schematic drawing of a first exemplary embodiment of a surgical gown.

FIG. 2 is a schematic drawing of a second exemplary surgical gown.

FIG. 3 is a schematic drawing of a third exemplary surgical gown.

FIG. 4 is a schematic drawing of a fourth exemplary surgical gown.

FIG. 5 is a schematic drawing of a fifth exemplary surgical gown.

FIG. 6 is a schematic drawing of a sixth exemplary surgical gown.

FIG. 7 is a schematic drawing of a seventh exemplary surgical gown.

FIG. 8 is an illustrative photograph of a surgeon wearing a surgical bouffant head covering in accordance with several embodiments.

FIG. 9 is an illustrative photograph of a surgeon wearing a surgical cap covering in accordance with several embodiments.

FIG. 10 is a schematic drawing of a first exemplary surgical glove.

FIG. 11 is a schematic drawing of a second exemplary surgical glove.

FIG. 12 is a schematic drawing of a third exemplary surgical glove.

FIG. 13 is a schematic drawing of a fourth exemplary surgical glove.

FIG. 14 is a perspective view of an inverted surgical glove on a glove former during the process of manufacturing the glove, illustrating the process of spraying a composition containing phase-change material onto the glove.

FIG. 15 is a perspective view of the surgical glove illustrated in FIG. 14 after the glove has been removed from the former.

FIG. 16 is a photograph of a woven material.

FIG. 17 is a photograph of a non-woven material.

FIG. 18 is an enlarged representational view illustrating a nonwoven gown material with microcapsules disposed on the surface of the gown and having penetrated the nonwoven gown fabric.

FIG. 19 is a photograph of an exemplary surgical gown.

FIGS. 20 and 21 are photographs of the interior of the surgical gown of FIG. 19.

FIGS. 22 and 23 are photographs of details of the interior of the surgical gown of FIG. 19.

FIGS. 24 and 25 are photographs of an exemplary bouffant cap.

FIG. 26 is a photograph of the interior of the surgical bouffant cap of FIGS. 24 and 25.

### DETAILED DESCRIPTION

Generally speaking, a disposable medical garment with cooling properties is disclosed herein. The medical garment may be used during operating procedures and the embodiments described herein are presented with respect to disposable surgical clothing, but it is contemplated that the clothing described herein is useful in other medical contexts. The medical garment may include, for example, a surgical gown, gloves, and a head covering that incorporates a phase change cooling agent. The particular phase change cooling agents described herein comprise microcapsules having a hydrophilic wall material. In addition to providing latent heat absorption and providing a cooling sensation to the wearer via this mechanism, it is believed that such hydrophilic microcapsules also may help disperse or wick moisture from the wearer by drawing dampness deeper into the non-woven fabric(s) that form the gown or backing material secured to the gown. Because nonwoven materials typically used in surgical garments are generally less breathable than woven fabrics, this moisture dispersion can assist in providing an additional cooling sensation for the wearer.

The medical garment may take many forms, including, for instance, a surgical gown, cap, or bouffant. For instance, in one illustrative configuration, a surgical gown includes a non-woven fabric forming a body for said a surgical gown. The body of the surgical gown includes a front torso section, a back section, and sleeve sections. The body defines an interior surface and an exterior surface, wherein the interior surface includes a phase change cooling agent deposited on a at least a portion thereof. In other forms, the medical garment takes the form of headwear such as a cap or bouffant, the headwear including a phase change cooling agent disposed on an inner surface thereof. For example, an exemplary bouffant cap includes a non-woven fabric forming a covering body with an elastic trim that is configured to rest on a head of a wearer adjacent the face and at least a portion of an interior of the covering body having a phase change cooling agent deposited thereon. In another configuration, a surgical cap includes, for example, a non-woven fabric forming a cap panel with a cap tie that is configured to secure the surgical cap to a wearer's head and at least a portion of the cap panel having a phase change cooling agent deposited thereon. It is also contemplated that the garment may take the form of a glove, the glove comprising an elastomeric body with conventional wrist, palm, and finger sections and having a cooling agent disposed on at least a portion of the glove.

By some approaches, the non-woven fabric used in the medical garment is formed of a laminated synthetic fabric web. In some illustrative configurations, the non-woven fabric is a spunbond and meltblown fabric. In some embodiments, the non-woven fabric is a spunbond-meltblown-spunbond fabric. For instance, conventional SMS material comprises a trilaminar non-woven fabric composed of a top layer of spunbond polypropylene, a middle layer of meltblown polypropylene, and a bottom layer of spunbond polypropylene. Other polymers are contemplated. The fabric may be treated with one or more liquid repellents to inhibit contamination with bodily and surgical fluids. When the garment takes the form of a glove, more typically the glove will be formed of an elastomeric material.

In any case, the cooling agent includes a plurality of microcapsules of a phase change material encapsulated in a hydrophilic wall material, and generally also includes a binder and a surfactant and an optional thickener. The phase change material should have a melting point that is greater than the typical operating room temperature or around 20° C. but no greater than normal body temperature, or 37° C., but may be lower, such as no greater than 36° C., no greater than 35° C., no greater than 34° C., no greater than 33° C., no greater than 32° C., no greater than 31° C., no greater than 30° C., no greater than 29° C., no greater than 28° C., no greater than 27° C., no greater than 26° C., no greater than 25° C., or no greater than 24° C. The phase change cooling agent may be disposed over the entire interior surface of the garment or only a portion of the interior surface but not the entirety thereof. In some illustrative approaches, the phase change cooling agent comprises microcapsules having a polyacrylate wall portion encapsulating a C₁₈ paraffin or wax, typically an alkane, having a melting point ranging from 28°-30° C. For certain applications, such as the rotogravure printing and other printing methods, the phase change cooling agent may be combined into a paste for application to the fabric, as described below.

When the garment takes the form of a surgical gown, such gown may include a torso portion that is disposed adjacent a wearer's trunk, the torso portion including a front torso section and a back torso section. The back of the gown may have an opening to enable donning of the gown, the opening being closeable with string ties, hook-and-loop strips, or the like that may be selectively secured to one another. Further, the surgical gown generally includes sleeves with cuffs and a collar configured to be worn adjacent a wearer's neck or collarbone. In some configurations, the cuffs and collar have elastic therein to configure portions of the garment to be worn closely to the wrist, neck, and/or collar bone. The gown may have a front drape portion and rear drape portion that cover the user's midsection and a portion of the user's legs.

In some embodiments, the phase change cooling agent is disposed directly on at least a portion the non-woven fabric that comprises the body of the gown. In other embodiments, the phase change agent is disposed on a backing sheet that is attached to the non-woven fabric that comprises the body of the gown. The backing sheet may be formed of a non-woven fabric material, such as a panel of the same material that comprises the gown, or may be formed of a woven material. Further, by some approaches, at least a portion of the microencapsulated phase change material may be disposed within the non-woven fabric that comprises the body of the gown or within the non-woven fabric that forms the backing material.

In some configurations, the phase change cooling agent may be rotogravure printed onto the non-woven fabric of the gown or onto a backing sheet that is attached to the non-woven fabric of the gown. In addition to being rotogravure printed, the phase change cooling agent may be applied via wooden blocks, stencils, engraved plates, silkscreens, rollers, or rotary screens, among others. This application method provides flexibility in both the depth of application and percentage of coverage. For example, the phase change cooling agent may have a pattern coverage of between about 10% to about 100%. In operation, the depth of the pattern in a printing roller ranges from between about 0.01 mm to about 10 mm, in some configurations. By some approaches, the depth is about 0.10 mm to about 0.20 mm. Further, the depth or thickness of the rotary nickel screen may range from between 0.05 mm to about 0.5 mm. By some approaches, the rotary nickel screen has a thickness of about 0.10 mm. Those of skill in the art understand that the rotary nickel screen or the roller would be etched with a desired pattern, which would then be filled with the paste having the phase change cooling agent therein, which is then transferred onto the fabric.

As noted, the phase change cooling agent may be combined into a paste for application to a fabric. By one approach, the paste includes an optional thickening agent, a surfactant, preferably a nonionic surfactant, a binding agent, and an encapsulated phase change cooling agent. The consistency of the phase change cooling agent paste may be adjusted to have a particular viscosity by adjusting the amount of a thickening agent present. One commercially available paste is sold as “PCMs-TCX” by Shanghai Geelong Chemical New Material Co., Ltd. (China).

In some embodiments, the nonionic surfactants include, for example, a polyethylene oxide surfactant (such as polyoxyethylene lauryl ether, polyoxyethylene cetyl ether, polyoxyethylene stearyl ether, polyoxyethylene oleyl ether, polyoxyethylene higher alcohol ether, polyoxyalkylene alkyl ether, and/or polyoxyethylene octyl phenyl ether, among others). Further, in some configurations, the nonionic surfactants include, for example, a polyol surfactant (such as sorbitan esters and/or sucrose ester, among others). The surfactant is used to enhance compatibility of the hydrophilic microencapsulated phase change cooling agent with the (often hydrophobic) binder materials. In addition, it is believed that the surfactant may cause some of the microcapsules to penetrate the surface of the nonwoven fabric that comprises the surgical garment or backing material, which is believed to be advantageous for dispersing moisture away from the user. The surfactant may be used in any suitable amount in the paste, such as an amount ranging from 0.5 to 20% by total weight of the paste.

In some configurations, the thickening agent includes, for example, polyurethanes, acrylic polymers, latex, styrene/butadiene, PVA, clays (such as attapulgite, bentonite, and/or montmorillonite, among others), cellulosics (such as CMC, HMC, and/or HPMC, among others), sulfonates (such as sodium and/or calcium salts, among others), gums (such as guar, xanthan, cellulose, locust bean, and/or acacia, among others), saccharides (such as carrageenan, pullulan, konjac, and/or alginate, among others), proteins (such as casein, collagen, and/or albumin, among others), organosilicones (such as silicone resins, dimethicones, and/or modified silicones, among others), and/or modified castor oil. The thickener is present to increase the viscosity of the paste to a level suitable for gravure printing or related printing techniques. When used, the thickener may be present in any suitable amount, such as an amount ranging from 0.1 to 40% by total weight of the paste.

The binder or binding agent may include, for example, acrylates and methacrylates (such as methyl acrylate, methyl methacrylate, ethyl acrylate, ethyl methacrylate, butyl acrylate, butyl methacrylate, 2-ethylhexyl acrylate, glycidyl acrylate, glycidyl methacrylate, hydroxyethyl acrylate, hydroxyethyl, methacrylate, hydroxypropyl acrylate, hydroxypropyl methacrylate, hydroxybutyl acrylate, hydroxybutyl methacrylate, and/or 3-chloro-2-hydroxybutyl methacrylate, among others). Further, the binding agent, in some configurations, includes, amino-functional acrylates (such as t-butyl amino methyl methacrylate and/or dimethylamino ethyl methacrylate) and dicarboxylic ester (such as alkyl and dialkyl fumarates, itaconates, and/or maleates having one to eight carbons, with or without functional groups, among others). For example, more specifically, the dicarboxylic esters may include diethyl and dimethyl fumarates, itaconates and maleates, di (ethylene glycol) maleate, di (ethylene glycol) itaconate, bis (2-hydroxyethyl) maleate, and 2-hydroxyethyl methyl fumarate. Further, natural binding agents may be incorporated into the paste, such as accroides, candelilla, guar, gum arabic, karaya, shellac, tragacanth, and/or xanthan, among others. The function of the binder is to adhere the phase change cooling onto the fabric upon which it is applied. The binder serves as the vehicle in the paste and generally should be present as the balance of the paste composition, although it is contemplated that the paste may include other ingredients such as preservatives or colorants, each of which may be present in any amount suitable for their respective purposes.

As described further below, the phase change cooling agent, in some configurations, may be applied as a spray, such as to gloves. In such a configuration, the phase change cooling agent may not be applied as a paste but instead may be sprayed in a format that lacks the thickening agent of the paste. As noted above, the thickening agent may be adjusted to adjust the viscosity, and therefore, removing or greatly reducing the presence of the thickening agent may be preferable for a spray application.

Further, the cooling garments also may include surgical gloves. By one approach, the disposable surgical glove includes a flexible body with a first area, a second area, and a third area. The first area includes a palm and back area that can be disposed adjacent either the palm or back of the hand. In one illustrative approach, the first area includes a designated palm-oriented surface and a designated back hand-oriented surface. The second area includes a plurality of finger-receiving receptacles. The third area includes a wrist-forearm area. The wrist-forearm area includes a cuff portion and may include a beaded cuff. In an exemplary configuration, the disposable glove is composed of a nitrile material and may also have a chlorinated coating or a partial chlorinated coating. In addition, as suggested above, the disposable surgical glove also may have a partially-treated interior that has a cooling agent disposed thereon. For example, the cooling agent that is partially-dispose on an interior of the disposable surgical glove may be found, for example, in the first area (i.e., the palm-back area), the second area (i.e., the finger receptacles) and/or the third area (i.e., the wrist and forearm area). Further, in some configurations, the surgical gloves include an interior partially treated with a phase change cooling agent and also have a beaded cuff that is resistant to glove cuff roll-down.

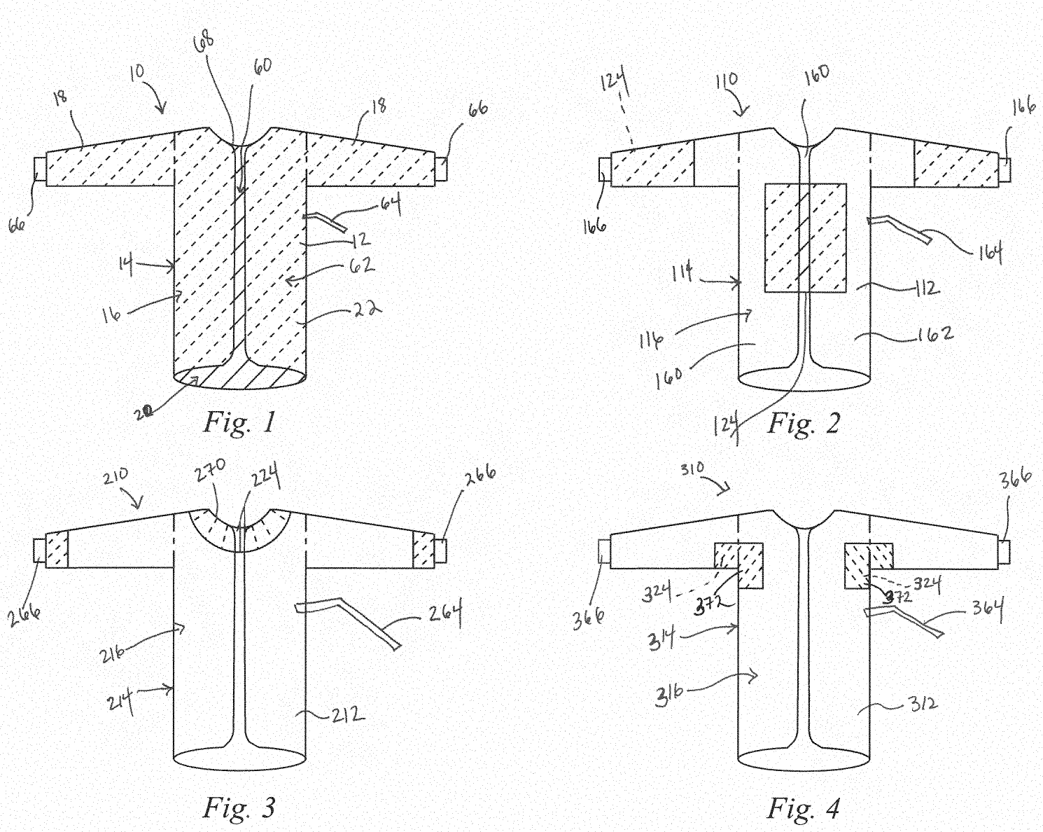

As shown in FIGS. 1-7, a variety of different surgical gowns 10, 110, 210, 310, 410, 510, 610 are illustrated with a phase change cooling agent 24 disposed in different configurations thereon. As shown for instance in FIG. 1, a surgical gown 10 includes a non-woven fabric 12 forming a body 14 having a trunk section 16, including a front torso 60 section and a back torso section 62, sleeves 18, and a donning slit or opening therein that is closable via one or more strings or ties 64. Further, the gown body includes an interior surface 20 and an exterior surface 22. The gown further includes a front drape portion 60 and rear drape portion 62 that cover respectively the user's midsection and a portion of the user's legs. Further, the surgical gown 10 also generally includes cuffs 66 near the ends of the sleeves 18 and a collar 68 configured to be worn adjacent a wearer's neck and/or collar bone. In some configurations, one or both of the cuffs 66 and collar 68 have elastic therein to snugly secure to the wrist, arm, neck, and/or collar bone.

The interior surface 20 includes a phase change cooling agent 24 deposited on a at least a portion thereof. While FIG. 1 illustrates the phase change cooling agent 24 over nearly the entirety of the interior surface 20 of the gown 10, other configurations, such as those illustrated in FIGS. 2-7, have the phase change cooling agent 24 partially disposed over the interior surface of the gown. Generally, a paste as heretofore described is applied to the fabric that forms the gown or to a backing sheet that is secured to the gown.

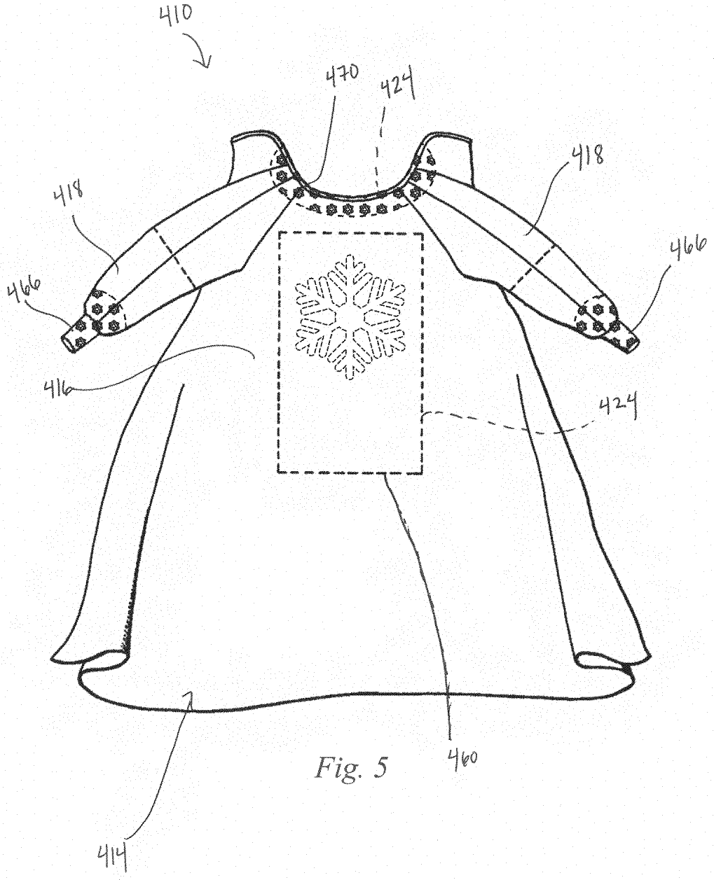

FIGS. 1-4 illustrate a rear schematic view of surgical gowns 10, 110, 210, 310 and show where the phase change cooling agent 24 is deposited via a diagonal line pattern. While FIG. 1 illustrates a surgical gown 10 that has a phase change cooling agent 24 continuously disposed on an entire interior surface 22 thereof, in other configurations, the phase change cooling agent 24 is disposed over only a portion of the interior surface but not the entirety thereof, as illustrated, for example, in FIGS. 2-7. While the phase change cooling agent 24 is shown via diagonal lines in FIGS. 1-4, the phase change cooling agent 24 may be printed in a variety of manners, such as in a picture or pattern, such as a snowflake pattern, as illustrated in FIGS. 5-7.

FIG. 2, which employs reference numerals similar to those from FIG. 1 with a 1- in front of the number, includes a surgical gown 110 with a gown body 114 having a trunk section 116 and sleeves 118. Further, the phase change cooling agent 124 is disposed on the sleeves 118 from the elbow section to the elastic cuffs 166. Further, the phase change cooling agent 24 is disposed on a front torso section 60 of the surgical gown 110.

FIG. 3, which employs reference numerals similar to those from FIG. 1 with a 2- in front of the number, includes a surgical gown 210 with a gown body 214 having a trunk section 216 and sleeves 218. Further, the phase change cooling agent 224 is disposed on the sleeves 218 adjacent the elastic cuffs 266. Further, the phase change cooling agent 24 is disposed on a portion of the gown body 214 adjacent the neck 270.

FIG. 4, which employs reference numerals similar to those from FIG. 1 with a 3- in front of the number, includes a surgical gown 310 with a gown body 314 having a trunk section 316 and sleeves 318. Further, the phase change cooling agent 324 is disposed on the interior of the gown body 314 at the underarm section 372.

FIG. 5 is similar to the previously discussed figures but illustrates the surgical gown 410 from the front thereof and employs reference numerals similar to those from FIG. 1 with a 4- in front of the number. As shown, the surgical gown 410 includes a gown body 414 having a trunk section 416 and sleeves 418. Further, the phase change cooling agent 424 is disposed on the interior of the gown body 314 adjacent the neck 470, adjacent the cuffs 466, and on a front torso portion 460.

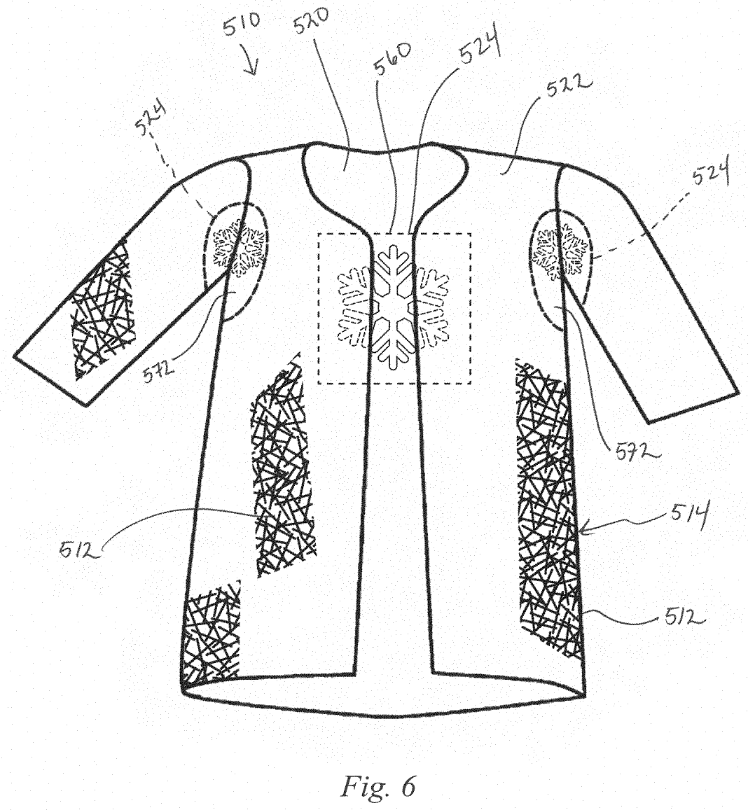

FIG. 6, which employs reference numerals similar to those from FIG. 1 with a 5- in front of the number, includes a surgical gown 510 with a gown body 514 having a trunk section 516 and sleeves 518. Further, the phase change cooling agent 524 is disposed on the interior of the gown body 514 at the underarm section 572 and on a front torso portion 560. Further, the gown body 514 also illustrates the non-woven fabric 512 forming the surgical gown 510.

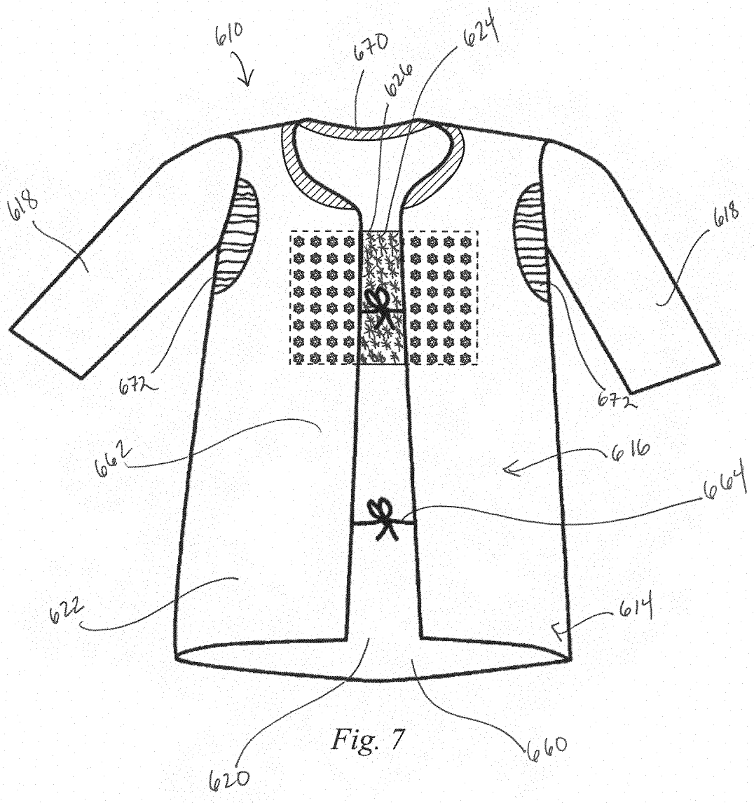

FIG. 7, which employs reference numerals similar to those from FIG. 1 with a 6- in front of the number, includes a surgical gown 610 with a gown body 614 having a trunk section 616 and sleeves 618. Further, the phase change cooling agent 624 is disposed on the interior of the gown body 614 at the underarm section 672, on a front torso portion 660, and adjacent the neck 670.

Having the phase change cooling agent 24 in a variety different areas and configurations may permit the surgeons to opt for a design that best suits their personal comforts.

As indicated above, the phase change cooling agent 24 may be disposed directly on at least a portion the non-woven fabric 12 that comprises the body 14 of the gown. Further, in other configurations, at least a portion of the microencapsulated phase change agent or material 24 is disposed within said non-woven fabric 12. In yet other configurations, the phase change agent 624 is disposed on a backing sheet 626 (FIG. 7) that is attached to the non-woven fabric 12 that comprises the body 14 of the gown. Further, the backing sheet 626 may be a non-woven fabric sheet and at least a portion of the microencapsulated phase change agent or material 624 may be disposed within the non-woven fabric sheet 612.

In some configurations, the surgical gowns 10, 110, 210, 310, 410, 510, 610 are formed of a laminated synthetic fabric web, such as a spunbond and meltblown fabric. In one illustrative approach, the non-woven fabric 12 is a spunbond-meltblown-spunbond trilaminar SMS polypropylene fabric.

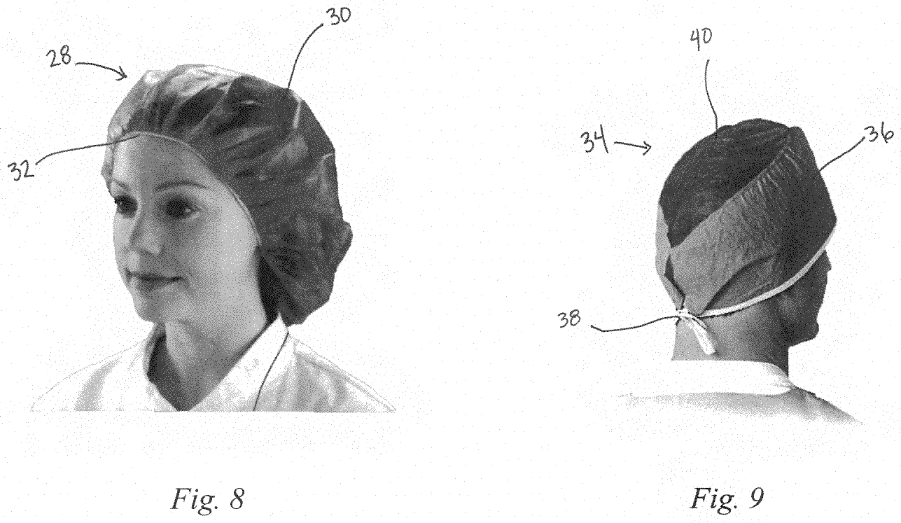

With reference to FIG. 8, the illustrated bouffant cap 28 includes a non-woven fabric 12 forming a covering body 30 with an elastic trim 32 configured to rest on a head of a wearer adjacent the face. Further, the bouffant cap 28 has at least a portion of an interior of the covering body having a phase change cooling agent 24 deposited thereon, the phase change cooling agent 24 being similar to that employed with the cooling surgical gowns discussed above. As with the gowns, the phase change cooling agent 24 may be disposed directly on the non-woven fabric that comprises the covering body, in some configurations, or may be applied to a separate backing sheet (not shown) that is then secured to the covering body.

FIG. 9 illustrates a surgical cap 34 of a non-woven fabric 12 forming a cap panel 36 with a cap tie 38 that is configured to secure the surgical cap to a wearer's head. Further, the surgical cap has at least a portion of the cap panel 36 having a phase change cooling agent 24 deposited thereon, the phase change cooling agent again being similar to the cooling agent described above. In some configurations, the surgical cap 34 includes top vent 40 that lacks the cooling agent 24. Further, similar to the cooling surgical gown and bouffant cap, the surgical cap 34 also may have a phase change cooling agent 24 disposed directly on the non-woven fabric that comprises the cap panel 36, or may be applied to a separate backing sheet (not shown) that is then secured to the covering body.

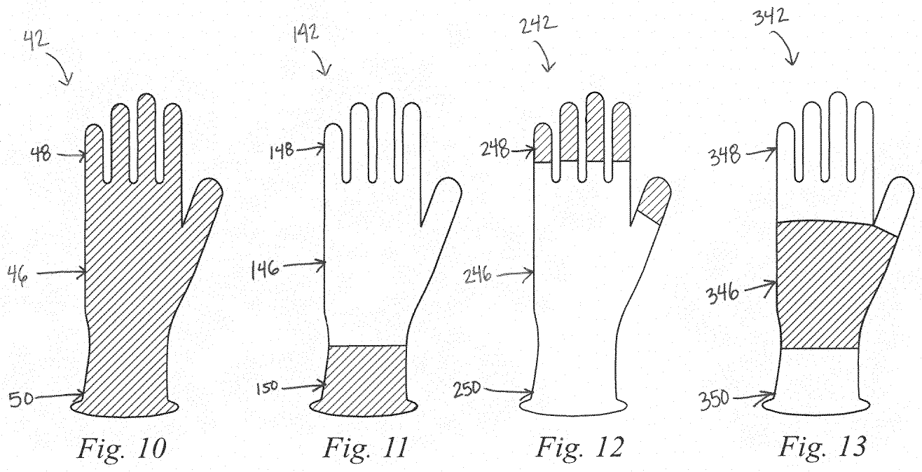

As noted above, the medical garments with the cooling properties may include gloves. FIGS. 10-13 illustrate a variety of different surgical gloves 42, 142, 242, 342 that have a phase change cooling agent 24 disposed in different configurations thereon. While FIG. 10 illustrates a surgical glove 42 that has a phase change cooling agent 24 continuously disposed on an entire interior surface thereof, in other configurations, the phase change cooling agent 24 is disposed over only a portion of the interior surface but not the entirety thereof, as illustrated, for example, in FIGS. 11-13. Further, while FIG. 10 illustrates the phase change cooling agent 24 over the entire interior surface thereof, in other configurations, the phase change cooling agent 24 may be disposed of nearly the entire interior surface, except for small portions of the interior, such as a bead at the end of the cuff.

As shown, the surgical glove 42, 142, 242, 342 typically includes a flexible body 44 with a first area 42, 142, 242, 342 including a palm-back area, a second area 48, 148, 248, 348 having a plurality of finger-receiving receptacles and a third area 50, 150, 152, 252, 352 forming a wrist and forearm area. By one approach, the flexible gloves have an inner surface and an outer surface, with the inner surface having a phase change cooling agent 24 deposited on at least a portion of the inner surface. Similar to those previously discussed, the cooling agent 24 applied to the interior of the surgical gloves may include a binder, a surfactant, and plural microcapsules of a phase change material encapsulated in a hydrophilic wall material, where the phase change material having a melting point no greater than 32° C. Where the embodiments employing a paste for application of the phase change cooling agent 24 require an thickener, the agent 24 mat be applied to the gloves via a spray technique and may therefore employ a thinner liquid.

While FIG. 10 illustrates the phase change cooling agent 24 over the entire inner surface of the glove 42, other configurations, such as those illustrated in FIGS. 11-13, have a partial coating of the phase change material over the glove interior. For example, FIG. 11 illustrates a phase change cooling agent 24 disposed in the third area 150 (e.g., adjacent the wearer's wrist and/or forearm), FIG. 12 illustrates a phase change cooling agent 24 disposed in a second area 248 (e.g., disposed adjacent the wearer's fingers), and FIG. 13 illustrates a phase change cooling agent 24 disposed in a first area 346 (e.g., disposed adjacent a wearer's palms or the back of the hand).

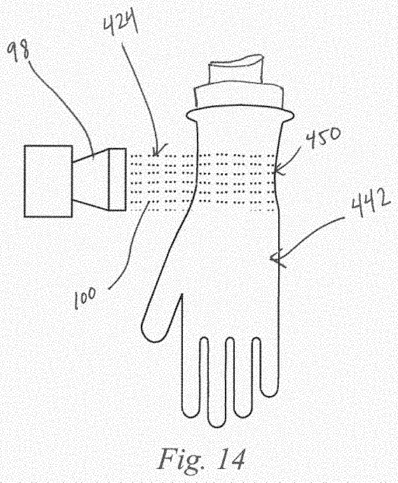

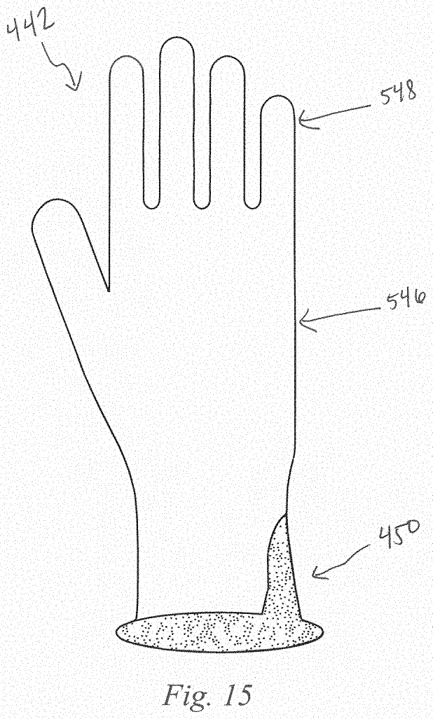

A method for making such disposable gloves generally comprises forming the disposable glove on a ceramic former. The disposable glove includes a first area, a second area, and a third area. In one illustrative approach, the first area includes a designated palm-oriented surface and a designated back hand-oriented surface. The second area includes a plurality of finger-receiving receptacles. The third area includes a wrist-forearm area. The method may include dipping the disposable glove into a solution to form the glove and then spraying the glove to deposit the phase change cooling agent 24 on a portion of the glove. This is illustrated generally in FIG. 14, showing inverted glove 442 being sprayed at wrist area 450 with a low-viscosity phase change cooling agent 424 that comprises a surfactant, binder, and microencapsulated phase change material 100 emitted from a nozzle 98. When removed from the former and inverted, as seen in FIG. 15, the glove 442 will have phase change cooling agent adhered to the glove in wrist area 450 but not in the palm or finger areas 546, 548 of the glove.

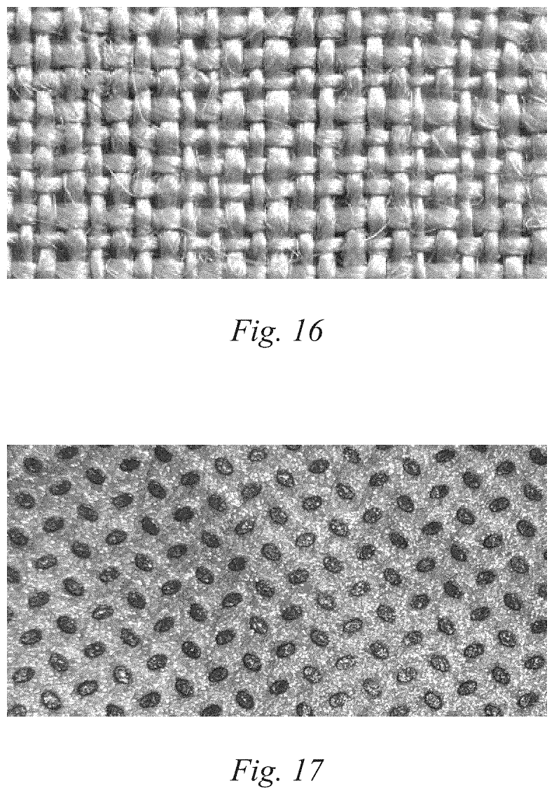

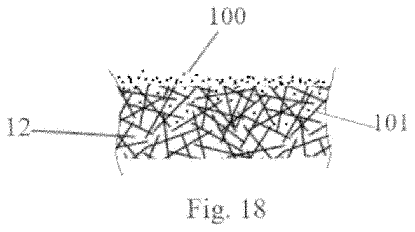

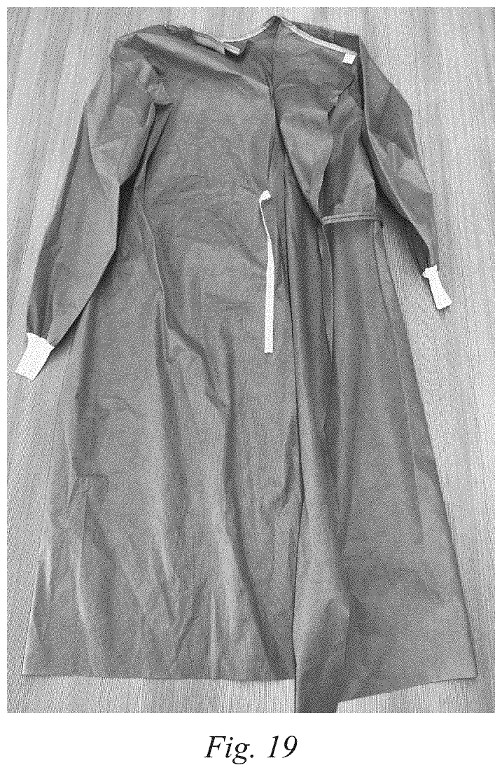

As seen by comparing FIGS. 16 and 17, a nonwoven material may be generally denser than a similarly sized woven material, with less interior porosity. Turning to FIG. 18, the nonwoven material 12 is composed of nonwoven fibers 101. Microcapsules 100 are disposed on the surface of the fibers and are bound thereto by the binder (not shown). Additionally, it is believed that the presence of the surfactant in the cooling agent enables some of the microcapsules to penetrate the surface of the fibers and to become entrapped within the nonwoven fibrous matrix. Again, this is deemed advantageous for assisting in drawing moisture away from the user and into the nonwoven material 12.

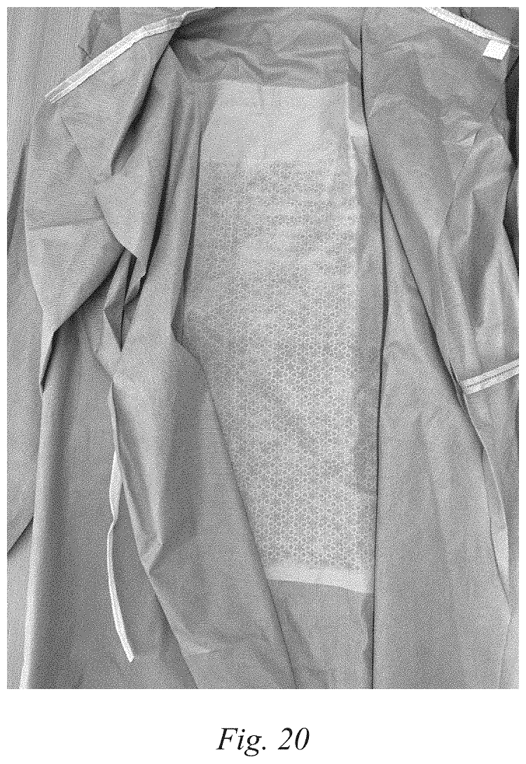

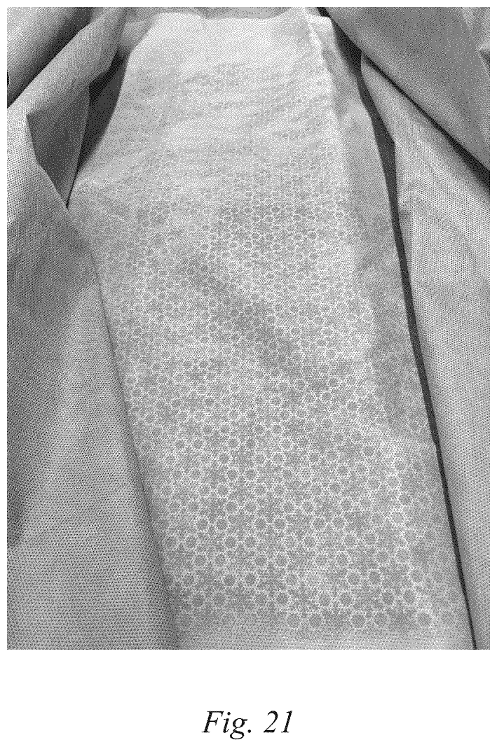

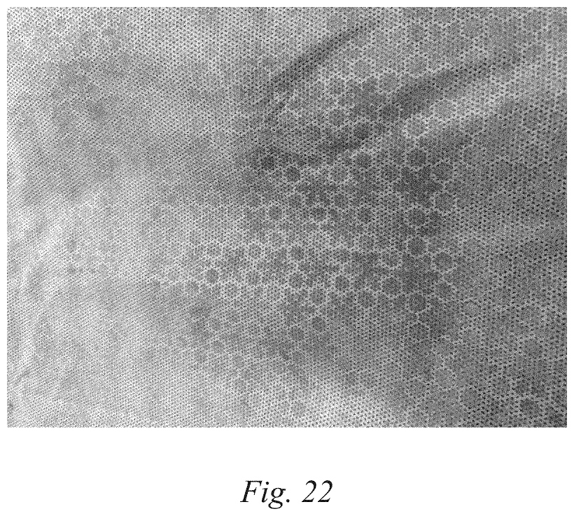

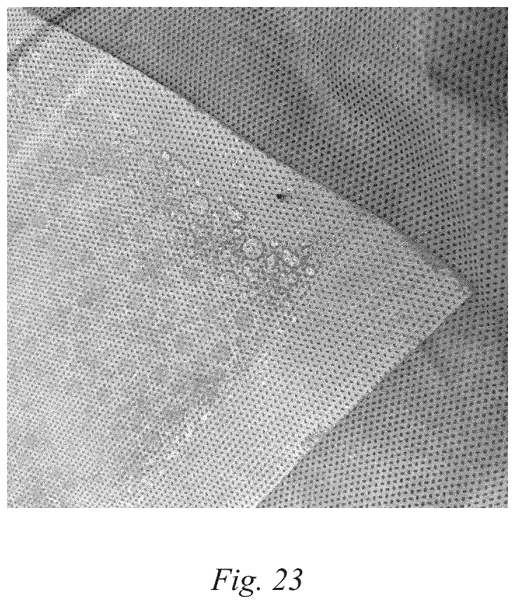

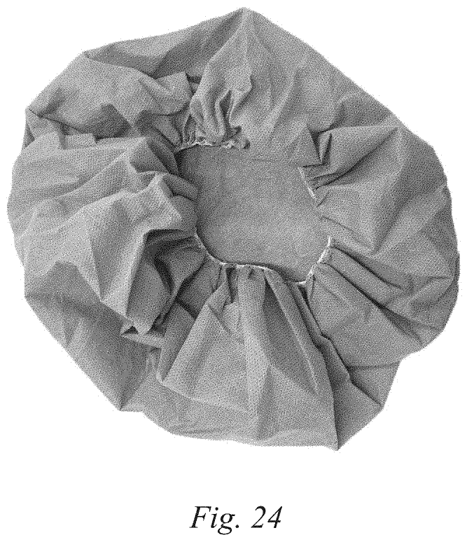

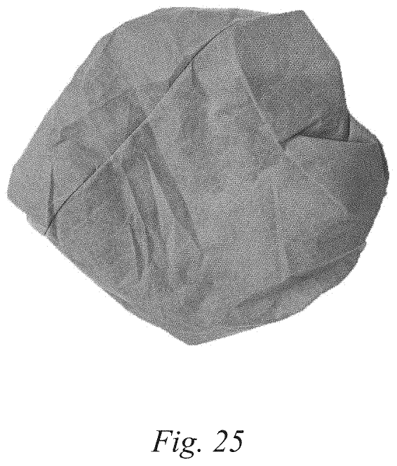

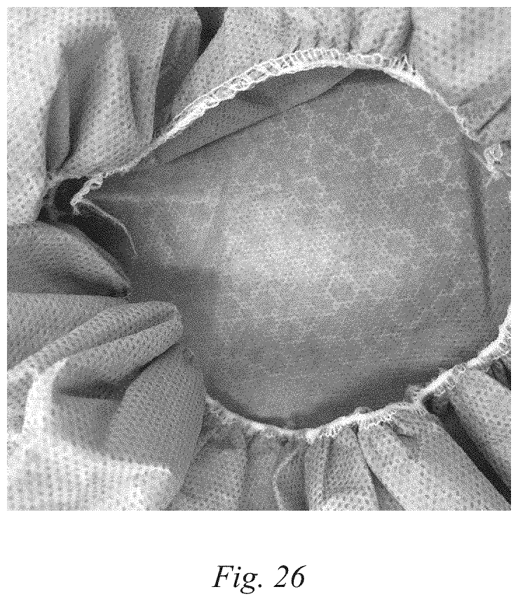

It is thus seen that a cooling agent may be incorporated into surgical clothing. Photographs of exemplary surgical clothing may be seen in FIGS. 19-26.

All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A surgical gown comprising:
a non-woven fabric forming a body for said a surgical gown, the body of the surgical gown having a front torso section, a back section, and sleeve sections, said body defining an interior surface and an exterior surface;
wherein the interior surface includes a phase change cooling agent deposited on a at least a portion thereof;
wherein the cooling agent includes a binder, a surfactant, and plural microcapsules of a phase change material encapsulated in a hydrophilic wall material, the phase change material having a melting point no greater than 32° C.

2. The surgical gown of claim 1, wherein the phase change cooling agent is disposed over only a portion of the interior surface but not the entirety thereof.

3. The surgical gown of claim 1 wherein the phase change cooling agent is disposed directly on at least a portion the non-woven fabric that comprises the body of the gown.

4. The surgical gown of claim 3 wherein at least a portion of the microencapsulated phase change material is disposed within said non-woven fabric that comprises the body of the gown.

5. The surgical gown of claim 1 wherein the phase change agent is disposed on a backing sheet that is attached to the non-woven fabric that comprises the body of the gown.

6. The surgical gown of claim 5 wherein the backing sheet is a non-woven fabric sheet.

7. The surgical gown of claim 6 wherein at least a portion of the microencapsulated phase change material is disposed within said non-woven fabric sheet.

8. The surgical gown of claim 1 wherein the phase change cooling agent comprises microcapsules having a polyacrylate wall portion encapsulating a C₁₈ wax.

9. The surgical gown of claim 1 wherein the cooling agent is rotogravure printed onto the non-woven fabric or onto a backing sheet that is attached to the non-woven fabric.

10. The surgical gown of claim 1 wherein the non-woven fabric is formed of a laminated synthetic fabric web.

11. The surgical gown of claim 1 wherein the non-woven fabric comprises a spunbond and meltblown fabric.

12. The surgical gown of claim 11 wherein the non-woven fabric comprises a spunbond-meltblown-spunbond fabric.

13. The surgical gown of claim 1 wherein the phase change cooling agent is disposed adjacent at least one of a wearer's trunk, neck, underarms, or wrists.

14. A surgical gown comprising:
a non-woven fabric forming a body for said a surgical gown, the body of the surgical gown having a front torso section, a back section, and sleeve sections, said body defining an interior surface and an exterior surface; and
a cooling agent printed onto the interior surface, wherein the cooling agent includes a binder, a surfactant, and plural microcapsules of a phase change material encapsulated in a hydrophilic wall material, the phase change material having a melting point no greater than 32° C.
wherein the cooling agent is pattern printed via a rotogravure printer.

15. A bouffant cap comprising:
a non-woven fabric forming a covering body with an elastic trim, the elastic trim configured to rest on a head of a wearer adjacent the face; and
at least a portion of an interior of the covering body having a phase change cooling agent deposited thereon, wherein the cooling agent includes a binder, a surfactant, and plural microcapsules of a phase change material encapsulated in a hydrophilic wall material, the phase change material having a melting point no greater than 32° C.

16. The bouffant cap of claim 15 wherein the phase change cooling agent is disposed directly on the non-woven fabric that comprises the covering body.

17. A surgical cap comprising:
a non-woven fabric forming a cap panel with a cap tie that is configured to secure the surgical cap to a wearer's head; and
at least a portion of the cap panel having a phase change cooling agent deposited thereon, wherein the cooling agent includes a binder, a surfactant, and plural microcapsules of a phase change material encapsulated in a hydrophilic wall material, the phase change material having a melting point no greater than 32° C.

18. The surgical cap of claim 17 further comprising a top vent that lacks the cooling agent.

19. The surgical cap of claim 18 wherein the phase change cooling agent is disposed directly on the non-woven fabric that comprises the cap panel.

20. A surgical glove comprising:
a flexible body forming the surgical glove, the flexible body having a first area including a palm-back area, a second area having a plurality of finger-receiving receptacles and a third area forming a wrist and forearm area, the flexible glove having an inner surface and an outer surface; and
a phase change cooling agent deposited on at least a portion of the inner surface;
wherein the cooling agent includes a binder, a surfactant, and plural microcapsules of a phase change material encapsulated in a hydrophilic wall material, the phase change material having a melting point no greater than 32° C.
